(19) United States
(12) Patent Application Publication
(10) Pub. No.: US 2021/0386445 A1
(43) Pub. Date: Dec. 16, 2021

(54) NASAL SMOKE EVACUATOR

(71) Applicants: John H. Burban, St. Paul, MN (US); Peter Joseph Catalano, Newton, MA (US); John W. Shanahan, White Bear Lake, MN (US); Michael R. Spearman, The Woodlands, TX (US); Craig J. Cuta, Stillwater, MN (US); Keith A. Roberts, Dellwood, MN (US)

(72) Inventors: John H. Burban, St. Paul, MN (US); Peter Joseph Catalano, Newton, MA (US); John W. Shanahan, White Bear Lake, MN (US); Michael R. Spearman, The Woodlands, TX (US); Craig J. Cuta, Stillwater, MN (US); Keith A. Roberts, Dellwood, MN (US)

(21) Appl. No.: 17/343,157

(22) Filed: Jun. 9, 2021

Related U.S. Application Data
(60) Provisional application No. 63/050,162, filed on Jul. 10, 2020.
(60) Provisional application No. 63/037,246, filed on Jun. 10, 2020.

(51) Int. Cl. A61B 17/24; A61M 1/00

(52) U.S. Cl. CPC A61B 17/24; A61B 2018/00327; A61B 2218/008; A61M 1/87

(57) ABSTRACT

A method and means effectively evacuates smoke plumes, vapors, aerosol release, mists, tissue and small particles produced from an operative nasal cavity and any other gas- or air-borne materials provided in or produced in the surgical environment. To this end, an evacuator tube is placed in the contralateral (suction) nasal cavity surrounded by a sealing member which blocks the space between the evacuator tube and nares and/or nasal vestibule.

### RELATED APPLICATIONS DATA

This application claims priority from two U.S. Provisional patent applications, U.S. Ser. No. 63/050,162, Filed 10 Jul. 2020 and Titled NASAL SMOKE EVACUATOR; and U.S. Ser. No. 63/037,246, filed 10 Jun. 2020, and Titled NASAL SMOKE EVACUATOR. These applications are incorporated herein in their entireties by reference.

### BACKGROUND OF THE INVENTION

### 1. Field of the Invention

The present invention relates to the field of surgery, especially aerosol generating surgical procedures where air-borne waste in the form of aerosols, smoke, particles, droplets and detritus are created and must be removed by mechanical or pneumatic systems, and especially during sinus surgical procedures.

### 1. Background of the Art

In a variety of surgical and dermatological procedures, diseased or other tissue is destroyed as part of the operative procedure. Many surgical procedures result in the creation of aerosols, smoke, droplets

In many cases, the tissue is destroyed by applying heat via electrosurgery or laser to the tissue causing the tissue to be burned away. This heat from this procedure typically vaporizes the intracellular fluid, bursting the cell membranes and results in a plume of smoke being produced at the operative site as the tissue is destroyed.

The health risks from inhaling the smoke generated during an operative procedure are not known. However, the smoke aerosols and particulates are small enough in size to potentially cause lung and eye irritation. The long-term effects of such irritation are also unknown. But it is known that the smoke aerosols and particulates may include viable microbial and viral contaminants as well as destroyed tissue.

This has led to the development and implementation of smoke evacuation systems being used during surgical procedures. In a current practice, a nurse or assistant holds a suction tube adjacent an operative site to suck in the plume. During the procedure, the nurse or assistant attempts to hold the suction nozzle near the operating site to collect as much of the generated plume as possible. The suction may be held in a fixed position close to the operative site with adhesive tape, clamps or other means. However, there is a great opportunity for some of the plume to escape in either method.

This is especially problematic in endoscopic sinus surgery/nasal surgery. Harmful airborne viruses and bacteria deposit on the nasal cavity and sinus tissues. When sinus surgery is performed, these can be become entrained in the plume. Endoscopic sinus surgery involves inserting an endoscope and surgical tools through the nostril of the patient. Trying to hold a suction close to the nostril during surgery is challenging as the surgeon needs room to move the endoscope and surgical tools, so this limits the amount of plume that can be captured.

Different surgical environments may require significantly different equipment to facilitate or protect the patient and the medical staff. Some procedures must be performed in ultra-clean rooms, others in ultra-sanitary rooms, and others must avoid emissions from patients, including smoke tissue and microbial-filled liquids. Newer methodologies such as using electrocautery, radio frequency, harmonic knives, lasers, drills and other instruments to cut or otherwise impact tissue can create aerosols and smoke within the nasal cavity cavity. The smoke may not only cause buildup, reduced visibility, or damage or desensitize equipment, but the air-borne residue may contain viable viruses or other infectious mass. Many different smoke evacuation systems have been developed over the years to try to capture and remove smoke generated from surgical procedures.

U.S. Pat. No. 4,701,193 (Robertson) discloses a smoke evacuator system for byproducts of laser surgery including an inline construction of a motor and a filter assembly mounted on opposite sides of a mounting plate. The filter fits in a filter duct and the temperature of the flow between the filter and the vacuum chamber of the vacuum pump can be monitored to determine the clog condition of the filter. The system includes electronic circuitry to activate an alarm device to indicate to the user that the filter is clogged and further circuitry is provided to shutoff the motor after a long period of operation with a clogged filter to prevent damage to the system by overheating.

U.S. Pat. No. 5,015,243 (Shifano) discloses a smoke evacuator that includes a head member that substantially surrounds an operative site to draw air and smoke from around a perimeter of the site as the smoke is produced. The head can be secured about the site during the procedure.

U.S. Pat. No. 5,226,939 (Nicolas) discloses a surgical air vacuum apparatus is includes an air pump disposed in a primary air channel for moving air between an inlet port and an outlet port of the vacuum. The air pump has properties for generating heat while moving the air, and the moving air has characteristics for cooling the pump. An air filter disposed in the primary channel has a tendency to block the air that would otherwise cool the air pump. Means is provided for combining a secondary air channel and the primary air channel along at least one passage to introduce bleed air to the pump when the filter blocks the primary channel. [Stackhouse]

U.S. Pat. No. 6,001,077 (Ellman) discloses a smoke evacuation apparatus designed to provide safe and efficient filtration and evacuation of smoke plume generated by laser-surgical, electrosurgical, radiosurgical, and electrocautery devices, and a wand for use therewith. The wand comprises at its free end a tapered tip across which is mounted a mesh configured to allow air to flow freely into the wand while at the same time being capable of capturing any solid objects entrained by the suctioned air. In a preferred embodiment, the tip is tapered on both sides, and meshes mounted across both tapered sides. This has the advantage that air can be suctioned from either or both sides of the tapered tip, and also allows the wand to be used as a tongue or tissue depressor without interfering with its ability to suction smoke and plume from the surgical site.

U.S. Pat. No. 6,458,125 (Cosmescu) discloses an electro-surgical unit (ESU) pencil apparatus is disclosed in which an improved handpiece enables the surgeon to vary the length of working electrode without having to use cutting electrodes of varying lengths. This is achieved by installing the electrode in an assembly that may, as desired by the surgeon, be extended from, or retracted into, the body of the ESU pencil apparatus. Means are provided for supplying uninterrupted RF supply to the electrode tip and uninterrupted smoke evacuation for the ESU pencil apparatus as the electrode assembly is expanded or retracted. Additionally, means are provided for locking the electrode assembly at the desired length, so that it does not move during the operation. The ESU pencil apparatus is also adapted for use with an ESU-argon beam coagulator pencil.

U.S. Pat. No. 7,207,977 (Thompson) discloses an improved smoke evacuation system and method for removing gaseous byproducts of surgical procedures and noxious vapors from chemicals is provided. The smoke and vapor evacuation system includes a vacuum head positionable at a surgical site or incorporated into a workstation. The vacuum head includes a plenum, and a plenum support for preventing the plenum from collapsing when a vacuum or low pressure is established therein, and is adapted to facilitate the use of the system in a variety of surgical or commercial procedures at a variety of surgical sites or commercial workstations.

U.S. Pat. No. 9,943,355 (Babini) discloses a smoke evacuation system for an open surgical site is disclosed which includes a filter cartridge assembly including a housing having an inlet port, a flow path extending from the inlet port, and a filter element disposed within the flow path, a flexible conduit having a proximal end connected to the inlet port of the filter assembly, and a smoke evacuator configured for detachable connection to a distal end of the flexible conduit for drawing smoke away from the surgical site and into the filter assembly through the conduit.

U.S. Pat. Nos. 6,110,259 and 6,589,316 (Schultz) discloses a smoke evacuating system for use during surgical procedures, particularly minimally invasive procedures involving the use of a laser or cautery at a surgical site having an associated higher than ambient pressure, wherein the system includes a filter with a site side and an outlet side and a fluid conduit extending between the surgical site and the filter. The filter exhibits low resistance or a low pressure drop and resists fluid flow, whereby the higher than ambient pressure is not substantially diminished and generates a fluid flow in the fluid flow path tending to carry smoke to and through the filter.

U.S. Pat. Nos. 8,414,550 and 8,608,715 (Roberts) disclose a method to vent gas from a closed body cavity during a laparoscopic procedure. A body cavity is in fluid communication with an exhaust gas inlet of a vacuum break device. The vacuum break device has a chamber in fluid communication with both the inlet and an outlet. The chamber may comprise one or more openings in fluid communication with the atmosphere. A conduit in fluid communication with the exhaust gas outlet may be connected directly or indirectly to a suction source. The suction source may be activated.

U.S. Pat. No. 9,821,095 (Lloyd) discloses a method to vent gas from a body cavity during an endoscopic procedure. A body cavity is in fluid communication with an exhaust gas inlet of a vacuum break device. The vacuum break device has a chamber in fluid communication with both the inlet and an outlet. The chamber may comprise one or more openings in fluid communication with the atmosphere. A conduit in fluid communication with the exhaust gas outlet may be connected directly or indirectly to a suction source. The suction source may be activated.

Published U.S. Patent Application Document 20200054799 (Wang) discloses a suction device for use with a medical device may include a suction device body and an extension. The suction device body may include a first lumen positioned therein and including a plurality of holes. The extension may include a second lumen positioned therein and may be coupled to the suction device body is and configured to facilitate connection of the suction device body to a suction line. The second lumen may be in communication with/open to the first lumen and the suction line may be adapted for connection to a pump configured to apply a negative pressure to the first and second lumens. The suction device may evacuate fluids and/or solids from a patient in an area proximate to the suction device.

Published U.S. Pat. No. 10,426,873 (Schultz) discloses an evacuation system and method for removing and/or treating gaseous and/or particulate byproducts of surgical procedures includes an end effector such as a side vent trocar, an operating room tower or the equivalent, a disposal vessel, a vacuum source and optionally a filter. The end effector may be coupled to the tower by a conduit, the tower may be coupled to the collection tank by a conduit, and the collection tank may be coupled to the vacuum source. Gaseous and/or particulate byproducts may flow from the surgical site through the trocar, conduits, tower tank and filter, and the flow may be regulated at least in part by the components of the system.

These systems are not easily used in sinus and nasal surgery. They are bulky, get in the way of the surgeon performing the surgery, require the purchase of new equipment to operate them. Also, most of these designs operate to capture aerosols or smoke by allowing them to leave the nasal cavity in a plume and then attempt to remove them from the air. This creates potential risks to the surgeon and operating staff if all the plume is not captured.

What is needed is a method for capturing all the plume that is generated during laparoscopic or sinus surgery to provide a safe environment for surgeons and staff.

### SUMMARY OF THE INVENTION

The present invention provides a method and means for effectively evacuating smoke plumes, vapors, aerosol release, mists, tissue and small particles is produced from an operative nasal cavity and any other gas- or air-borne materials provided in or produced in the surgical environment. To this end, the invention includes an evacuator tube that is placed in the contralateral (suction) nasal cavity surrounded by a sealing member which blocks the space between the evacuator tube and nares and/or nasal vestibule. The evacuator tube is attached to a vacuum source during the operative procedure to create the necessary suction from the operative nasal cavity (the nasal cavity on which surgery is being performed) to the contralateral (suction) nasal cavity, to effectively capture and remove aerosols, smoke plumes, vapors, tissue and small particles produced from the operative nasal cavity.

One embodiment includes a system and method remove aerosols and smoke from aerosol generating surgical procedures, the method including:

providing an aerosol evacuator comprising an elongate flexible polymeric evacuator tube;
attaching a sealing member to the evacuator tube, the sealing member having a proximal side and a distal side, and an open central lumen;
fitting the sealing member into the nares of the nose to create a tight seal between the sealing member and nares or nasal vestibule;
performing the surgery in the nasal cavity, sinus, mouth, throat, larynx or upper gastrointestinal area of a patient having the nasal passage and generating smoke from the surgery in a volume in communication with the nasal passage; [the internal area may be the nasal cavity, larynx, mouth, throat, upper GI tract]
applying reduced gas pressure (vacuum) to the proximal end of the evacuator tube; and
withdrawing smoke, aerosols, droplets and other particles resulting from the surgery out of the patient.

Another embodiment includes a system and method remove aerosols and smoke from aerosol generating surgical procedures, the method including:

providing an aerosol evacuator comprising an elongate flexible polymeric evacuator tube;
placing a sealing member over the evacuator tube, the sealing member having a proximal side and a distal side;
a distal end of the evacuator tube passes through the sealing member, and the distal end of the evacuator tube extending from 0-15 cm past the distal side of the sealing member;
inserting the distal end of the evacuator tube into a nasal cavity during or before the surgery;
fitting the sealing member into the nares of the nose to create a tight seal between the sealing member, evacuator tube and nares or nasal vestibule;
performing the surgery in the nasal cavity, sinus, mouth, throat, larynx or upper gastrointestinal area of a patient having the nasal passage and generating smoke from the surgery in a volume in communication with the nasal passage; [the internal area may be the nasal cavity, larynx, mouth, throat, upper GI tract]
applying reduced gas pressure (vacuum) to the proximal end of the evacuator tube; and
withdrawing smoke, aerosols, droplets and other particles resulting from the surgery out of the patient.

### BRIEF DESCRIPTION OF THE FIGURES

FIG. 1 shows a side, perspective view of a device intended to be inserted into the nasal cavity according to one aspect of the present invention.

FIG. 2 shows a side view of the device of FIG. 1 inserted within a nasal cavity.

FIG. 3A shows a variation in the shape and openings in the distal tip used with the flexible evacuator tube to reduce clogging of the tip.

FIG. 3B shows a variation in the shape and openings in the distal tip used with the flexible evacuator tube to reduce clogging of the tip.

FIG. 3C shows a variation in the shape and openings in the distal tip used with the flexible evacuator tube to reduce clogging of the tip.

FIG. 3D shows a variation in the shape and openings in the distal tip used with the flexible evacuator tube to reduce clogging of the tip.

FIG. 3E shows a variation in the shape and openings in the distal tip used with the flexible evacuator tube to reduce clogging of the tip.

FIG. 3F shows a variation in the shape and openings in the distal tip used with the flexible evacuator tube to reduce clogging of the tip.

FIG. 4 shows a cross section view of a device intended to seal in the nares or nasal cavity according to one aspect of the present invention.

FIG. 5A shows a variation in the shape and opening of the sealing member.

FIG. 5B shows a variation in the shape and opening of the sealing member.

### DETAILED DESCRIPTION OF THE INVENTION

A system and method to remove smoke, (aerosols, droplets, or other particles during sinus surgery (this may be laser heat, probe heat, resistive electrical heat, abrasive, flame or friction heat and or any other procedure which generates aerosols, droplets or other particles). In one embodiment of the invention, the method includes providing an aerosol evacuator having an elongate tube. The tube may be flexible or rigid. It may be made of polymeric or metal materials.

A sealing member is connected to the evacuator tube, the sealing member having a proximal side and a distal side. The proximal end of the tube must be long enough to reach a connection to a vacuum source. The sealing member has a central lumen.

The sealing member is inserted into the nares during or before the surgery, which is to be done on an interior portion of the patient's body, in a cavity in gas-fluid communication with the nasal cavity where the evacuator tube is inserted. The sealing member should form a tight fit with the nares or nasal vestibule to create an air flow reduction seal between the sealing member and a nose having the nasal passage.

In another embodiment of the invention, the method includes providing an aerosol evacuator having an elongate tube. The tube may be flexible or rigid. It may be made of polymeric or metal materials. A sealing member is located over the evacuator tube, the sealing member having a proximal side and a distal side. A distal end of the tube passes through the sealing member, and the distal end of the tube extending from 0-15 cm past the distal side of the sealing member. The distance may be preset, or adjusted as desired by sliding the seal distally or proximally on the tube, and the proximal end must be long enough to reach a connection to a vacuum source.

The distal end of the evacuator tube is inserted into a nasal passage (through the nares) during or before the surgery, which is to be done on an interior portion of the patient's body, in a cavity in gas-fluid communication with the nasal cavity where the evacuator tube is inserted.

The sealing member should form a tight fit with the nares or nasal vestibule while over the evacuator tube to create an air flow reduction seal between the sealing member and a nose having the nasal passage.

The surgery is performed on an internal area of a patient having the nasal passage and generating smoke from the surgery in a volume in gas-fluid communication with the nasal passage. Reduced gas pressure (vacuum) is applied to and through to the proximal end of the evacuator tube. Smoke (and attendant aerosols, droplets, liquid and some small residue such as tissue) and other solids resulting from the surgery are suctioned out of the patient's nasal cavity and removed. The evacuator tube can be made of plastic, metal, rubber, elastomer, paper or other materials and made for a surgical environment where it is preferably biocompatible and does not have its own residue that would be incompatible with the surgical environment. Silicone polymers, vinyl polymers, nylon polymers, elastomeric polymers, polymeric plastics, stainless steel, and the like, as known in the art, may be used.

The sealing member may be constructed from a compressible solid material or foam material 25 that can expand and conform its outer surface to the anatomy of the nostrils, nares or vestibule of the nose.

The compressible elastomeric or foam material that can conform and seal to the nares or nasal vestibule and may, for example, be selected from the group consisting of a polyurethane, silicone, synthetic elastomer, vinyl, poly vinyl chloride, rubber and or other materials know in the art for creating seals. The foam is preferably a closed cell foam or an open celled foam whose faces are essentially nonporous, as a reticulated foam may allow flow to enter through the suction side and reduce the vacuum and flow from the operative side. Soft durometer and flexible materials (like soft O-rings, or gums) rather than hard durometer or stiff materials (like rigid plastic or a hockey puck) are preferable to be used to enhance conformation to the shape of the nares or nasal vestibule and stabilize the vacuum and for patient comfort. Foam materials may also be used, again desiring softer, flexible foams rather than stiff foams like a swimming practice kickboard. Open celled foams can be used in this invention, however they will require a stronger suction to generate the same air flow in the contralateral nasal passage. Likewise, the sealing member does not necessarily need to make a leak free seal with the nares, as leaks can be overcome with increased suction levels.

Reducing of the pull-weight of the evacuator tube and sealing member away from the nasal passage may be performed by a stabilizing method selected from the group consisting of adhesive strips securing the evacuator tube to the patient, clothes of the patient and local medical apparatus; fabric-lock strips securing the evacuator tube to the patient, clothes of the patient and local medical apparatus; and ties that secure the evacuator tube to the patient, clothes of the patient and local medical apparatus. Standard medical tapes, cloth tapes, paper tapes (for hypoallergenic needs) and the like may be used. The securing elements may even be distinct slide-on elements, such as a sleeve with Velcro® fasteners, snaps, hooks, pins and the like to connect with the patient's clothing or wraps.

The sealing member may have a slight taper on the distal side of the sealing member to provide easer insertion through the nostril, or a protuberance to better seal with a nasal vestibule of the nose of the patient.

The surgery may be any type of surgery within or adjacent to a nasal cavity. The method may be performed with evacuator tube inserted into one sinus/nasal cavity while the surgery is performed on the adjacent sinus/nasal cavity or the mouth, larynx, throat or upper gastrointestinal tract. The method may be performed with the aerosol evacuator tubes inserted in each nasal cavity when surgery is performed on the mouth, larynx, throat or upper gastrointestinal tract to provide higher suction and flow rates, or in just one nasal cavity with an occlusive device in the other nasal cavity or nares.

The suction pressure should be sufficient to withdraw smoke droplets out of the patient from the adjacent nasal cavity. Note that although smoke is emphasized in the disclosure, the present invention provides a method and means for effectively evacuating smoke plumes, vapors, aerosols, mists, tissue and small particles produced from an operative nasal cavity and any other gas- or air-borne materials provided in or produced in the surgical environment.

In one embodiment within the generic scope of the present invention, the structure may include:
A flexible, water-insoluble tube with the distal end of the tube extending into the nasal cavity (e.g., of a mammalian or human patient);
A sealing member which occupies the space between the tube and the nares and/or nasal vestibule;
The proximal end of the tube attaching to a vacuum source.

In another embodiment within the generic scope of the present invention, the structure may include:
A flexible, water-insoluble tube with the distal end of the tube connecting to a sealing member;
A sealing member which occupies the space between the tube and the nares and/or nasal vestibule and has a central lumen;
The proximal end of the tube attaching to a vacuum source.

Other features within the scope of the present invention which may be included in the practice of this technology are:

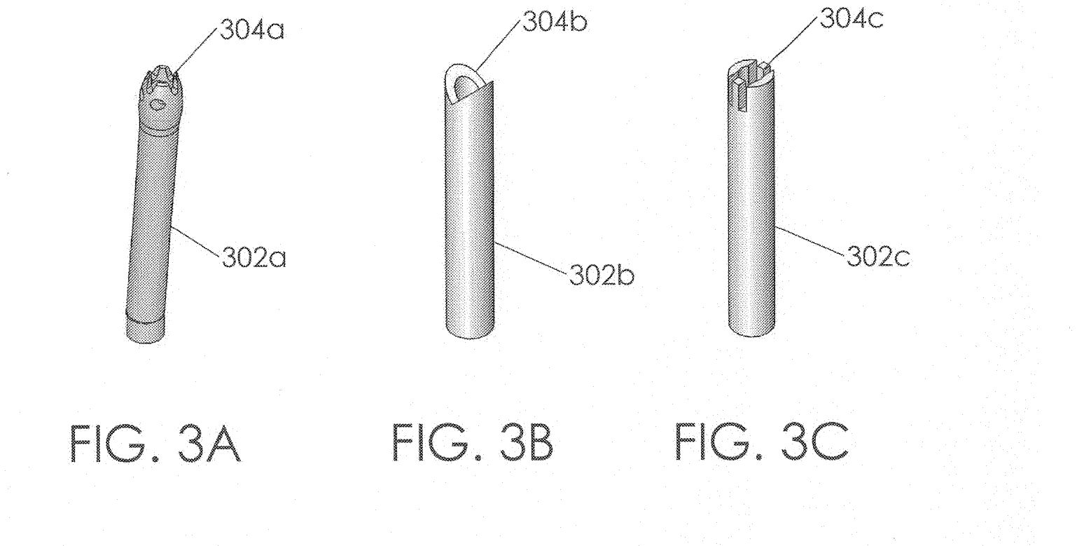

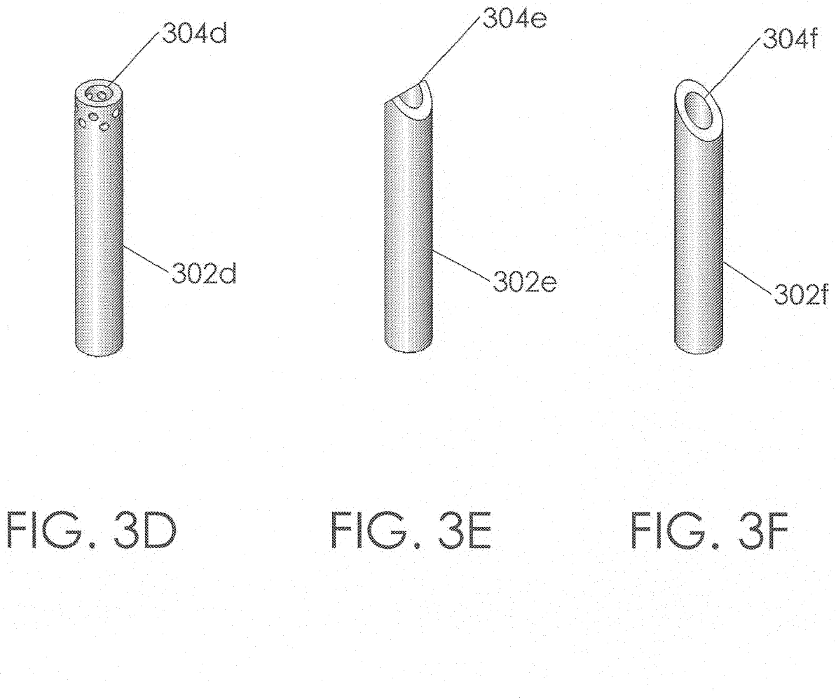

The distal end of the tube extending into the sinus/nasal cavity, preferably to the nasopharynx, to prevent evacuated aerosols, droplets and tissue particles from depositing in the contralateral sinus/nasal cavity or escaping into the operating room. FIGS. 3A, 3B, 3C, 3D, 3E, and 3F show variations on the tip of the evacuation tube that can maintain suction in the event the distal end of the tube becomes occluded.

One advantage of the system of the present invention is that gas-carried materials such as the smoke or droplets produced during a sinus operation is safely and immediately removed from an operative site within the nasal cavity, mouth, throat or larynx. It is not allowed to escape outside of the nose or mouth and become entrained in the surgical field and operating room.

Another advantage of the present invention is that, because smoke or tissue can be removed from the operative site, inhalation of such smoke is prevented or substantially reduced.

Yet another advantage of the invention is that the device is easily held in place within the contralateral nasal cavity, thus eliminating the need for one to hold a suction tube in the proximity of the operative site.

Yet another advantage of the invention is that it does not consume any space in the operative nasal cavity which already contains the endoscope and surgical instruments.

These and other advantages will become apparent from the following description of the preferred embodiments thereof, taken in conjunction with the accompanying drawings.

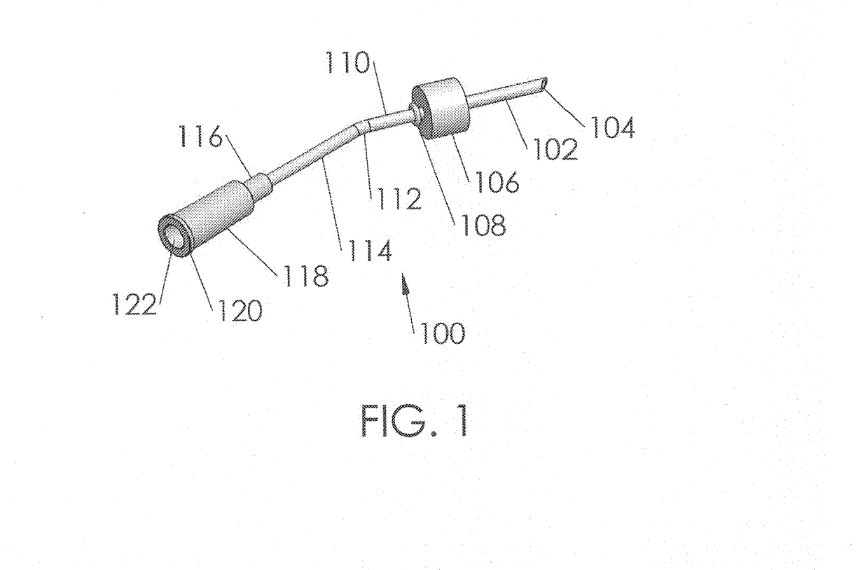

FIG. 1 shows a side, perspective view of a device 100 intended to be inserted into the nasal cavity according to one aspect of the present invention. The device 100 is shown with the distal end of the evacuator tube 102 having an optionally angled cut 104 to assist directing suction out of the distal end of the evacuator tube 102. A cushioning, preferably conformable sealing element 106 is prevented from sliding up the proximal end of the evacuator tube 102 by a permanently positioned or slidable barrier element 108 on the proximal side of the sealing element 106. The barrier element 108 also assists the surgeons in gauging how deep into the nasal cavity the distal end of evacuator tube 102 is placed. A more intermediate and more proximal segment 110 of the sealing element 106 extends from the sealing element 106 to a connecting (at an angle, straight or articulating) joint 112 connecting to an even more proximal evacuator tube segment 114. The more proximal evacuator tube segment 114 engages a series 116 and 118 of connectors with a proximal collar or washer (preferably elastomeric to form a tight seal) 120 creating an engagement opening 122 through which the vacuum is applied.

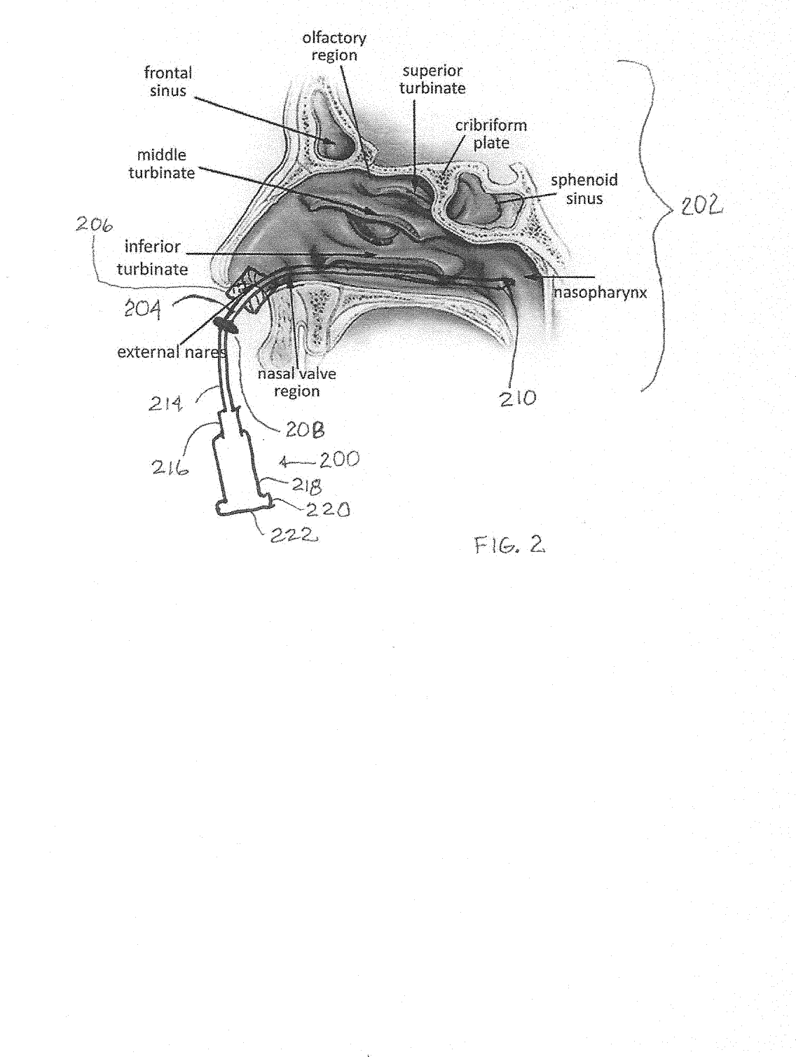

FIG. 2 shows a side view of the device of FIG. 1 200 inserted within a nasal cavity 202 (entire range of anatomy). The device 200 is shown with the distal end of the evacuator tube 204 having been extended from the nares into the nasopharanx. A conformable sealing element 206 is shown sealingly engaging the nares. A permanently positioned or slidable barrier element 208 on the proximal side of the sealing element 206 is shown providing a gauge for the depth of insertion of the evacuation tube 210. A more proximal evacuator tube segment 214 engages a series 216 and 218 of connectors with a distal collar or washer (preferably elastomeric to form a tight seal) 220 creating an engagement opening 222 through which the vacuum is applied.

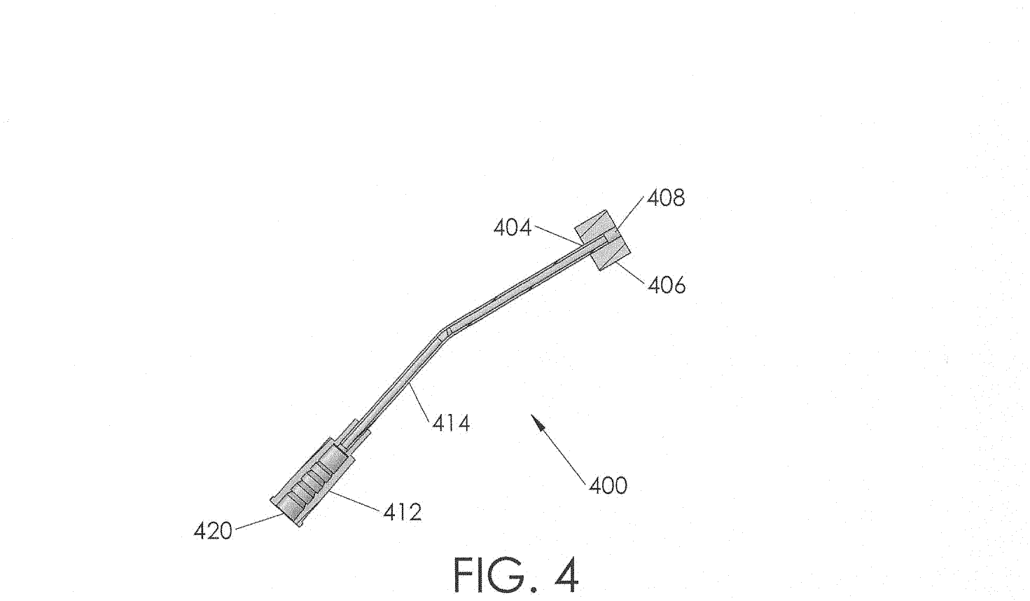

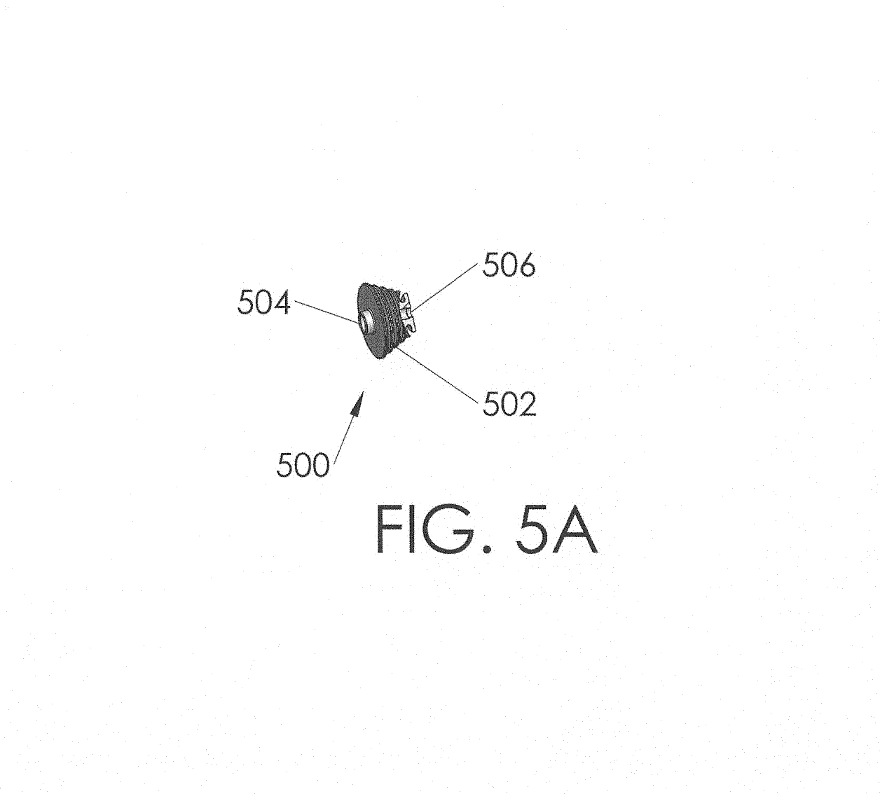

FIG. 4 shows a cross sectional view of a device 400 intended to be inserted into the nares or vestibule of a nasal cavity according to one aspect of the present invention. The device 400 is shown with the distal end of the evacuator tube 404 attaching to the sealing element 406 which then in use sealingly engages with the nares. The sealing element 406 contains a through hole 408. A more proximal evacuator tube segment 414 engages a connector 412 (preferably elastomeric to form a tight seal) creating an engagement opening 420 through which the vacuum is applied.

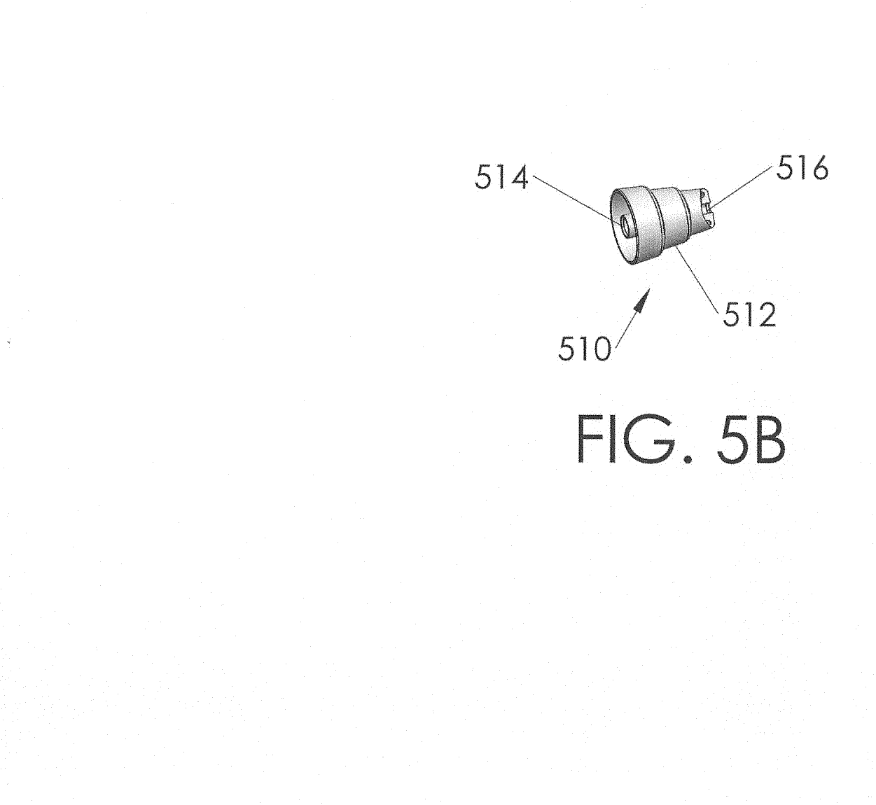

FIG. 5A and FIG. 5B shows other embodiments of the sealing members which sealingly engage with the nares. FIG. 5A shows a sealing member 500 with concentric wings 502, a connection point 504 for the evacuator tubing and a through hole 506. FIG. 5B shows a sealing member 510 with a tapered stopper design of the wings 512, a connection point 514 for the evacuator tubing and a through hole 516. In both of these designs, the wings are flexible and conformable to the nares and the depth of insertion of the sealing member would be based on the patient's nasal anatomy.

These and other aspects of the invention are recited in the claims.

What is claimed:

1. A method of removing aerosols and particles from aerosol generating surgical procedures, the method comprising:
providing an evacuator comprising an elongate evacuator tube;
placing a sealing member over the evacuator tube, the sealing member having a proximal side and a distal side;
a distal end of the evacuator tube passes through the sealing member, and the distal end of the evacuator tube extending from 0-15 cm past the distal side of the sealing member;
inserting the distal end of the evacuator tube into a nasal passage during or before the surgery;
the sealing member occupying the space between the evacuator tube and the nares or nasal vestibule to create an air flow reduction seal between the sealing member and nares or nasal vestibule;
applying vacuum to the distal end of the evacuator tube; and
withdrawing smoke, aerosols, droplets and/or other solids resulting from the surgery out of the patient through the evacuator tube.

2. The method of claim 1 wherein the evacuator tube comprises rigid tubing.

3. The method of claim 1 where the evacuator tube comprises flexible tubing.

4. The method of claim 1 where the evacuator tube comprises rigid and flexible tubing

5. The method of claim 1 wherein the sealing member comprises a compressible polymeric or foam material that can conform to the nares or nasal vestibule.

6. The method of claim 3 wherein the compressible polymeric or foam material is selected from the group consisting of a polyurethane, silicone, elastomer, vinyl, rubber and open or closed-cell foam.

7. The method of claim 1 wherein the reducing of the pull-weight of the aspirator tube and sealing member away from the nasal passage is performed by a stabilizing method selected from the group consisting of adhesive strips securing the aspirator tube to the patient, clothes of the patient and local medical apparatus; fabric-lock strips securing the aspirator tube to the patient, clothes of the patient and local medical apparatus; and ties that secure the aspirator tube to the patient, clothes of the patient and local medical apparatus.

8. The method of claim 4 wherein the reducing of the pull-weight of the aspirator tube and sealing member away from the nasal passage is performed by a stabilizing method selected from the group consisting of adhesive strips securing the aspirator tube to the patient, clothes of the patient and local medical apparatus; fabric-lock strips securing the aspirator tube to the patient, clothes of the patient and local medical apparatus; and ties that secure the aspirator tube to the patient, clothes of the patient and local medical apparatus.

9. The method of claim 1 wherein the sealing member has a protrusion on the distal side of the sealing member to better seal with a nasal vestibule of the nose of the patient.

10. The method of claim 1 wherein the surgery is an aerosol generating surgical procedure of a nasal cavity.

11. The method of claim 1 wherein the aerosol generating surgical procedure is electrocautery, laser ablation, drilling, radiofrequency, harmonic cutting, cryotherapy, or coblation.

12. The method of claim 1 wherein the aerosol evacuator tube is inserted into one nasal cavity while the surgery is executed on the adjacent nasal cavity.

13. The method of claim 1 wherein the aerosol evacuator tube is inserted into one or both nasal cavities while the surgery is executed on the mouth, larynx, throat or upper gastrointestinal cavities.

14. The method of claim 10 wherein the evacuator tube is inserted into one nasal cavity while the surgery is executed on an adjacent nasal cavity.

15. The method of claim 1 wherein the reduced pressure or vacuum is sufficient to withdraw smoke and liquids out of the patient from the adjacent nasal cavity.

16. The method of claim 12 wherein the reduced pressure or vacuum is sufficient to withdraw smoke and liquids out of the patient from the adjacent nasal cavity.

17. The method of claim 13 wherein the reduced pressure or vacuum is sufficient to withdraw smoke and liquids out of the patient from the mouth, larynx, throat or upper gastrointestinal cavities.

18. The method of claim 1 wherein performing the surgery is implemented in an internal area of a patient having the nasal passage and generating aerosols from the surgery in a volume in communication with the nasal passage.

19. The method of claim 3 wherein performing the surgery is implemented in an internal area of a patient having the nasal passage and generating aerosols from the surgery in a volume in communication with the nasal passage.

20. A surgical device for removal of aerosol particulates or droplets comprising: a device having a distal end of an evacuator tube, a sealing element having a barrier element to restrain movement of the sealing element along the evacuator tube.

21. The surgical device of claim 20 having a more proximal evacuator tube segment that a connector with a proximal over an engagement through which the vacuum is applied.

22. A method of removing aerosols and particles from aerosol generating surgical procedures, the method comprising:
providing an evacuator comprising an elongate evacuator tube;
providing a sealing member with a central lumen, the sealing member having a proximal side and a distal side;
a distal end of the evacuator tube attaches to the sealing member;
inserting the sealing member into a nasal passage during or before the surgery;
the sealing member occupying the space between the nares or nasal vestibule to create an air flow reduction seal between the sealing member and nares or nasal vestibule;
applying vacuum to the distal end of the evacuator tube; and
withdrawing smoke, aerosols, droplets and/or other solids resulting from the surgery out of the patient through the evacuator tube.

22. The method of claim 22 wherein the distal end of the evacuator tube is extended from 0-15 cm past the distal side of the sealing member.
